(19) United States
(12) Patent Application Publication
(10) Pub. No.: US 2009/0197895 A1
(43) Pub. Date: Aug. 6, 2009

(54) OXAZOLE-PYRROLE-PIPERAZINE ALPHA-HELIX MIMETIC

(75) Inventors: Julius Rebek, JR., La Jolla, CA (US); Shannon Biros, Grand Haven, MI (US); Lionel Moisan, Gif-Sur-Yvette (FR); Severin Odermatt, Zurich (CH)

Correspondence Address: DARBY & DARBY P.C., P.O. BOX 770, Church Street Station, New York, NY, 10008-0770, US

(73) Assignee: The Scripps Research Institute, La Jolla, CA (US)

(21) Appl. No.: 12/362,934

(22) Filed: Jan. 30, 2009

Related U.S. Application Data
(60) Provisional application No. 61/063,128, filed on Jan. 31, 2008.

(51) Int. Cl. A61K 31/496; C07D 413/14; C40B 40/04

(52) U.S. Cl. 514/254.02; 544/369; 506/15

(57) ABSTRACT

Amphiphilic α-helix mimetics are provided. These compounds are constructed using an oxazole-pyrrole-piperazine (OPP) scaffold. The amphiphilic α-helix mimetics are also employable for making libraries and for treating diseases or conditions effected by the inhibition or disruption of interactions with the alpha helix of a protein.

### CROSS-REFERENCE TO RELATED APPLICATIONS

The present application claims the benefit of U.S. Provisional Patent Application Ser. No. 61/063,128, filed Jan. 31, 2008, herein incorporated by reference in its entirety.

### GOVERNMENT RIGHTS

The invention described herein was supported in part by grant numbers GM50174 and GM27932 from the National Institutes of Health. The government has certain rights to this invention.

### FIELD OF INVENTION

The present invention relates to compounds, intermediates and methods for the preparation and uses thereof, and pharmaceutical compositions comprising the compounds. More particularly, the invention relates to oxazole-pyrrole-piperazine (OPP) scaffolds employable as amphiphilic α-helix mimetics and to their synthesis and use.

### BACKGROUND

α-Helices are key secondary structures involved in protein-protein recognition, with side chains in positions i, i+3/i+4, and i+7 at the interaction interface (Davis, J. M.; et al. Chem. Soc. Rev. 2007, 36, 326). Since the early 90's, attempts to mimic the spatial disposition of the side chains of an α-helix have been reported (Horwell, D. C.; et al. Tetrahedron 1995, 51, 203; Nolan, W. P.; et al. Tetrahedron Lett. 1992, 33, 6879), but only during the past few years has much effort been devoted to the design and synthesis of such mimetics (Davis, J. M.; et al. Chem. Soc. Rev. 2007, 36, 326; Fletcher, S.; Hamilton, A. D. J R. Soc. Interface 2006, 3, 215; Yin, H.; Hamilton, A. D. Angew. Chem. Int. Ed. 2005, 44, 4130; Jain, R.; et al. Mol. Divers. 2004, 8, 89; Peczuh, M. W.; Hamilton, A. D. Chem. Rev. 2000, 100, 2479). The best of them are the developments by Hamilton and co-workers that led to non-peptidic α-helix mimetics able to efficiently disrupt protein-protein interactions (Becerril, J.; Hamilton, A. D. Angew. Chem. Int. Ed. 2007, 46, 4471; Yin, H.; et al. J. Am. Chem. Soc. 2005, 127, 5463; Ernst, J. T.; et al. Angew. Chem. Int. Ed. 2003, 42, 535; Ernst, J. T.; et al. Angew. Chem. Int. Ed. 2002, 41, 278; Orner, B. P.; et al. J. Am. Chem. Soc. 2001, 123, 5382) such as Bak/Bcl-X_{L} (Yin, H.; et al. J. Am. Chem. Soc. 2005, 127, 10191; Kutzki, O.; et al. J. Am. Chem. Soc. 2002, 124, 11838) or p53/HDM2 (Yin, H.; et al. Angew. Chem. Int. Ed. 2005, 44, 2704). These molecules are based on terphenyl and related scaffolds which can mimic the spatial position and angular orientation of amino acid side chains on one “face” of an α-helix. As part of our own efforts in the synthesis of protein-protein interaction inhibitors (Davis, C. N.; et al. Proc. Natl. Acad. Sci. USA 2006, 103, 2953; Bartfai, T.; et al. Proc. Natl. Acad. Sci. USA 2004, 101, 10470; Bartfai, T.; et al. Proc. Natl. Acad. Sci. USA 2003, 100, 7971), we have developed methodology for the synthesis of amphiphilic α-helix mimetics featuring more hydrophilic components (Biros, S. M.; et al. Bioorg. Med. Chem. Lett. 2007, 17, 4641; Volonterio, A.; et al. Org. Lett. 2007, 9, 3733; Moisan, L.; et al. Heterocycles 2007, 73, 661); Mann, E.; et al. Tetrahedron Lett. 2008, 49, 903-905)).

### SUMMARY

One aspect of the invention is directed to a non-peptidic alpha-helix mimetic represented by Formula I:

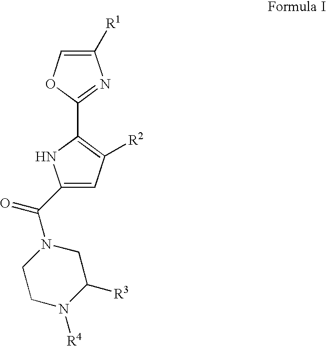

In Formula I, R¹, R² and R³ are independently selected from the group of radicals consisting of —H, C₁-C₆ alkyl, C₆-C₁₂ aryl; C₇-C₁₈ alkylaryl, C₄-C₁₈ alkylheterocycle, C₇-C₁₈ alkylheteroaryl, wherein one —CH₂— of the alkyl may be replaced by —S—, and wherein the alkyl, aryl, or heteroaryl may be substituted with one or two moieties selected from the group consisting of C₁-C₃ alkyl, OH, SH, NH₂, and COOH.

In Formula I, R⁴ is selected from the group of radicals consisting of —H, —C₁-C₆ alkyl, —C(O)O(C₁-C₆ alkyl), C(O)O(C₆-C₁₂ aryl), —C(O)O(C₇-C₁₈ alkylaryl), —C(O)O(C₇-C₁₈ alkylheteroaryl), —SO₂(C₁-C₆ alkyl), —SO₂(C₆-C₁₂ aryl), —SO₂(C₇-C₁₈ alkylaryl), —SO₂(C₇-C₁₈ alkylheteroaryl), —C(O)NH(C₁-C₆ alkyl), —C(O)NH(C₆-C₁₂ aryl); —C(O)NH(C₇-C₁₈ alkylaryl), and (C₇-C₁₈ alkylheteroaryl), wherein one —CH₂— of the alkyl may be replace by —S—, and wherein the alkyl, aryl, or heteroaryl may be substituted with one or two moieties selected from the group consisting of C₁-C₃ alkyl, OH, SH, NH2, and COOH.

In one aspect of the invention, the compound of Formula I is represented by the compound of Formula II:

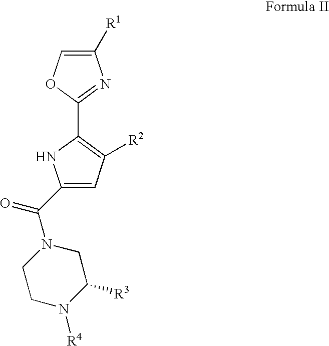

Compounds of the present invention also include pharmaceutically acceptable salts of the compounds of Formula I or Formula II. In one aspect of the invention the pharmaceutically acceptable salt is in the ammonium ion form.

In one embodiment, the compounds of Formula I or Formula II are selected from compounds wherein R¹, R² and R³ are independently selected from the group of radicals consisting of —CH₃, —CH₂CH₃, —CH(CH₃)₂, —CH₂CH₂CH₃, —CH₂CH(CH₃)₂, —CH₂CH(CH₃)CH₂CH₃, —CH(CH₃)CH₂CH₃, —CH₂CH₂CH₂CH₃, —CH₂CH₂CH(CH₃)₂, -Ph, —CH₂Ph, —CH₂CH₂Ph, —CH₂(1-naphthyl), —CH₂CH₂(1-naphthyl), —CH₂(2-naphthyl), —CH₂CH₂(2-naphthyl), —CH₂(3-indolyl), —CH₂CH₂(3-indolyl), —CH₂C₆H₄OH, —CH₂CH₂C₆H₄OH, —CH(OH)CH₃, —CH₂OH, —CH₂SH, —CH₂CH₂SH, —CH₂CH₂SCH₃, —CH₂CH₂CH₂SCH₃, —CH₂(4-imidazolyl), and —CH₂CH₂(4-imidazolyl); and R⁴ is selected from the group of radicals consisting of —H, —(C₁-C₆ alkyl), —C(O)O(C₁-C₆ alkyl), —C(O)O(CH₂aryl), —SO₂(C₁-C₆ alkyl), —SO₂aryl, —SO₂(CH₂aryl), —C(O)NH(C₁-C₆ alkyl), —C(O)NH(CH₂aryl), and —C(O)NH(aryl) or a pharmaceutically acceptable salt.

In another embodiment, the compounds of Formula I or Formula II are selected from compounds wherein R¹ is an alkyl or an aryl. In another embodiment, R² is an alkyl, aryl, or alkylaryl. In another embodiment, R³ is H or an alkyl.

In one embodiment, one —CH₂— of the alkyl portion of the alkylaryl, alkylheterocycle, or alkylheteroaryl of R¹, R², R³ or R⁴ may be replace by —S—. In another embodiment, the alkyl portion of the alkylaryl, alkylheterocycle, or alkylheteroaryl of R¹, R², R³ or R⁴ is a straight chain, unsubstituted alkyl without an optional —S— replacement.

In one embodiment, the alkyl, aryl, or heteroaryl of R¹, R², R³ or R⁴ is substituted with one or two moieties selected from the group consisting of C₁-C₃ alkyl, OH, SH, NH₂, and COOH. In another embodiment, the alkyl of R¹, R², or R³ is optionally substituted with OH or SH and the aryl of R¹, R², or R³ is optionally substituted with OH.

In one aspect of the invention, a preferred subgenus of this aspect of the invention is represented by the following structure:

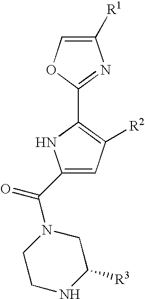

or a pharmaceutically acceptable salt. Particular species of the above subgenus are represented by the following structures:

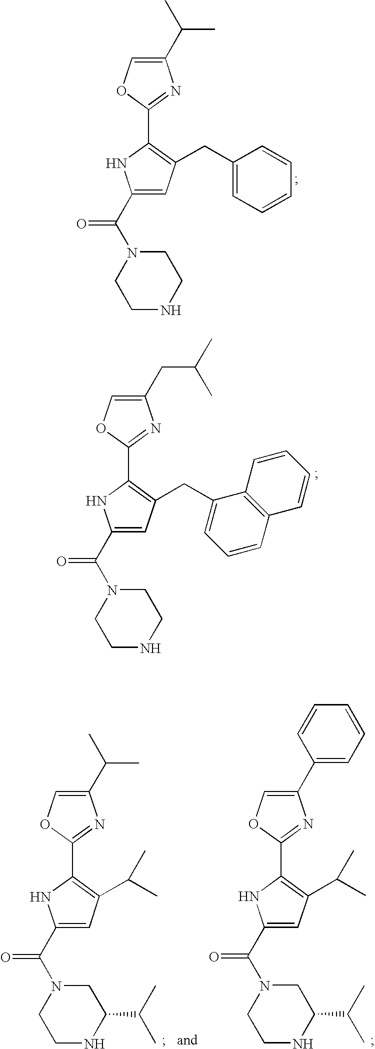

or a pharmaceutically acceptable salt thereof. In one embodiment, the pharmaceutically acceptable salt comprises an ammonium ion.

Another aspect of the invention is directed to a library of non-peptidic alpha-helix mimetics. The library comprises a collection of compounds represented by Formula I:

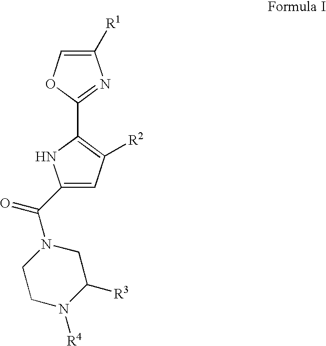

wherein R¹, R², R³ and R⁴ are as discussed above, or a pharmaceutically acceptable salt thereof.

In one embodiment, the library comprises a collection of compounds represented by Formula II:

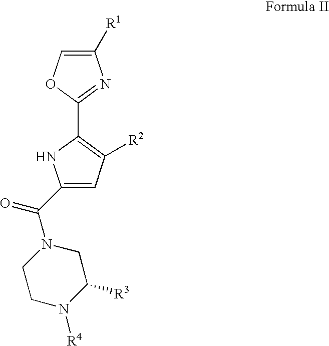

wherein R¹, R², R³ and R4 are as discussed above, or a pharmaceutically acceptable salt thereof.

Yet another embodiment of the present invention includes a pharmaceutical compound comprising the compound of Formula I or Formula II as discussed above or a pharmaceutically acceptable salt thereof and a pharmaceutically acceptable excipient.

The compounds of Formula I and/or Formula II are useful as amphiphilic alpha-helical mimetics for efficiently disrupting protein-protein interactions such as Bak/Bcl-X_{L}, p53/HDM2, calmodulin/smooth muscle myosin light-chain kinase, and gp41 assembly. Thus, yet another embodiment is a method of inhibiting or disrupting the interactions between an alpha helix of a first protein and an alpha helix binding pocket of a second protein, by contacting a compound of Formula I or Formula II as discussed above or a pharmaceutically acceptable salt thereof with the first protein and the second protein under conditions wherein the interactions between the alpha helix of the first protein and the alpha helix binding pocket of the second protein are inhibited or disrupted.

Methods for treating diseases or conditions which are modulated through disruption of interactions between alpha-helical proteins and their binding sites are other aspects of the invention.

### BRIEF DESCRIPTION OF DRAWINGS

FIG. 1a illustrates an overlay of 1 with a generic α-helix (Molecular modeling and compound superimpositions were carried out using the HyperChem™ 7.51 program).

FIG. 1b illustrates the general retrosynthetic approach to the scaffold 1.

FIG. 2 illustrates a table showing exemplary dienophiles that are capable of reacting with the dimethyl 1,2,4,5-tetrazine dicarboxylate 5, the temperature of the reaction, the yield and the structure of the product obtained.

FIG. 3 illustrates a scheme showing a strategy toward the synthesis of the OPP scaffold.

FIG. 4 illustrates a scheme showing asynthetic route for the synthesis of the OPP scaffold.

### DETAILED DESCRIPTION

Alkyl means a straight-chain or branched-chain aliphatic hydrocarbon radical having 1 to 12 or more preferably 1 to 6 carbon atoms. Suitable alkyl groups include, but are not limited to, the linear alkyl radicals methyl, ethyl, propyl, isopropyl, butyl, sec-butyl, tert-butyl, pentyl, and hexyl. Other examples of suitable alkyl groups include, but are not limited to, the substituted linear alkyl radicals 1-, 2- or 3-methylbutyl, 1,1-, 1,2- or 2,2-dimethylpropyl, 1-ethylpropyl, 1-, 2-, 3- or 4-methylpentyl, 1,1-, 1,2-, 1,3-, 2,2-, 2,3- or 3,3-dimethylbutyl, 1- or 2-ethylbutyl, ethylmethylpropyl, trimethylpropyl, and the like. In one embodiment, the alkyl may be a cyclic alkyl.

Aryl refers to an aromatic carbocyclic radical containing 6 to 14 carbon atoms, preferably 6 to 12 carbon atoms, especially 6 to 10 carbon atoms. Exemplary aryls are phenyl and naphthyl.

Heteroaryl refers to an aromatic carbocyclic radical containing 3 to 12 carbon atoms, preferably 3 to 8 carbon atoms, and at least one heteroatom selected from O, N, and S. The heteroaryl may be monocyclic or bicyclic. Exemplary heteroaryls radicals include pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, and the like. In one embodiment, the heteroaryl is an indolyl, pyrrolyl, or imidazolyl.

Heterocycle refers to a non-aromatic, cyclic radical containing 3 to 12 carbon atoms, preferably 3 to 8 carbon atoms, and at least one heteroatom selected from O, N, and S. The heterocycle may be monocyclic or bicyclic. Exemplary heterocycles radicals include pyrrolidinyl.

In some embodiments the alkyl, aryl, or heteroaryl may be substituted. Substituted radicals preferably have 1 to 3 substituents, or they may have 1 to 2 substituents which may be the same or different. In one embodiment, a single substituent is present. Preferred substitutions include C₁-C₃ alkyl, —OH, —SH, NH₂, —CO₂, and —C(O)NH.

Alkylaryl means an alkyl having at least one alkyl hydrogen atom replaced with an aryl moiety, such as —CH₂-phenyl, —CH₂CH₂CH₂-phenyl, and —CH₂-naphthyl.

Alkylheterocycle means an alkyl having at least one alkyl hydrogen atom replaced with a heterocycle moiety, such as —CH₂-pyrrolidinyl and —CH₂CH₂-pyrrolidinyl.

Alkylheteroaryl means an alkyl having at least one alkyl hydrogen atom replaced with a heteroaryl moiety, such as —CH₂-indolyl, —CH₂CH₂CH₂-indolyl, and —CH₂-pyrimidinyl.

As used herein, disrupting an interaction between a first protein and a second protein refers to the process of perturbing one or more covalent or non-covalent bonding interactions between the first and the second protein. Covalent bonding interactions between proteins include, for example, disulfide bonds, ester bonds, amide bonds and the like. Non-covalent bonding interactions between proteins include, for example, hydrophobic interactions, van der Waals interactions, ionic interactions, hydrogen bonding interactions and the like.

As used herein, inhibiting an interaction between a first protein and a second protein refers to the process of lowering the overall ability of the two proteins to bind or associate.

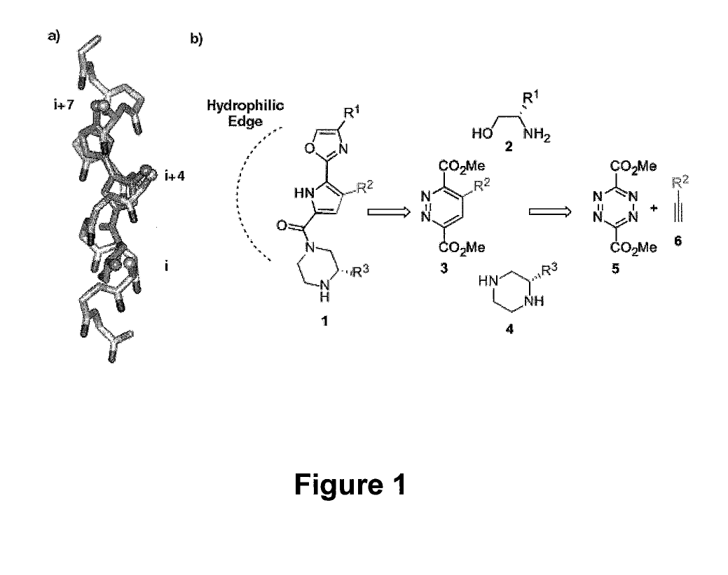

A general depiction of the target molecules described herein is shown in FIG. 1b along with an overlay of an α-helix (FIG. 1a). These molecules feature a linear arrangement of oxazole, pyrrole and piperazine units and may be thought of as synthetic counterparts to amphiphilic α-helices. The scaffold is intended to present both a hydrophobic surface for recognition and a hydrophilic edge that is rich in hydrogen bond donors and acceptors.

The general retrosynthetic approach is laid out in FIG. 1b. The major disconnections from the final OPP compound 1 are made at the amide bonds to give a pyridazine diester 3 that is the precursor to the pyrrole, an amino alcohol 2 and a piperazine unit 4. This synthesis is modular and involves mostly amide bond-forming reactions after the initial cycloaddition. Many amino alcohols as well as a variety of Boc-protected 2-substituted piperazines bearing standard hydrophobic side chains are commercially available. The central pyridazine ring is readily available from the inverse electron demand Diels-Alder reaction of known dimethyl-1,2,4,5-tetrazine-dicarboxylate 5 and a suitable dienophile (For reviews see: Boger, D. L. Chem. Rev. 1986, 86, 781; and Boger, D. L. Tetrahedron 1983, 39, 2869). The oxazole-pyrrole substructure can also be found in some marine alkaloids such as the Phorbazole family (Loughlin, W. A.; et al. Aust. J. Chem. 1999, 52, 231).

The modular synthesis of the α-helix mimetics based on an oxazole-pyrrole-piperazine scaffold has been performed as described herein. This scaffold is intended to present both a hydrophobic surface for recognition and a hydrophilic edge that is rich in hydrogen bond donors and acceptors. While specific derivatives are prepared and disclosed here, the methodology reported is applicable for a broader, more general decoration of the scaffold to provide a diversity of compounds. A library of compounds that bear common hydrophobic amino acid side chains was synthesized with this strategy for evaluation of their effects on various protein-protein interactions.

### DETAILED DESCRIPTION OF FIGURES

FIG. 1a shows an overlay of 1 with a generic α-helix (Molecular modeling and compound superimpositions were carried out using the HyperChem™ 7.51 program. Structures were minimized using the MM+forcefield and then the AM1 semi empirical method. The final rendering was obtained with WebLabViewerPro 4.0).

FIG. 1b shows the general retrosynthetic approach to the scaffold 1. The major disconnections from the final oxazole-pyrrole-piperazine (OPP) compound 1 are made at the amide bonds to give a pyridazine diester 3 that is the precursor to the pyrrole and the piperazine unit 4 and the amino alcohol 2. The pyridazine diester 3 is in turn derived from the inverse-electron demand Diels-Alder cycloaddition between dimethyl-1,2,4,5-tetrazine dicarboxylate 5 and a suitable dienophile 6.

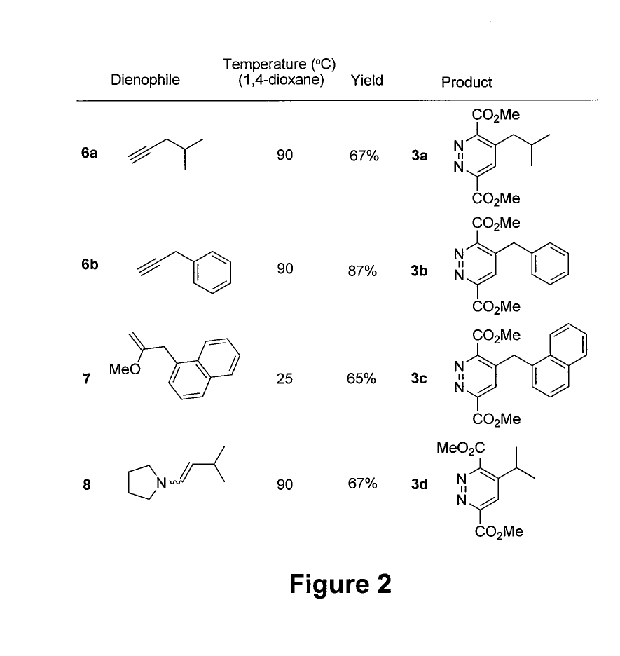

FIG. 2 is a table showing exemplary dienophiles that are capable of reacting with the dimethyl 1,2,4,5-tetrazine dicarboxylate 5, the temperature of the reaction, the yield and the structure of the product obtained. Alkynes, enol ethers and enamines are all usable dienophiles for the cycloaddition reaction. The first two dienophiles, the dienophile 6a, 4-methyl-pentyne, and 6b, 3-phenylpropane, are commercially available and were used in the cycloaddition reaction. The enol ether 7 was obtained by reaction of the methyl ester, methyl 1-naphthaleneacetate, with the Tebbe reagent. The naphthalene unit is used to mimic the indole side chain of tryptophan. Enamine 8 is obtained by condensation of pyrrolidine with isovaleraldehyde.

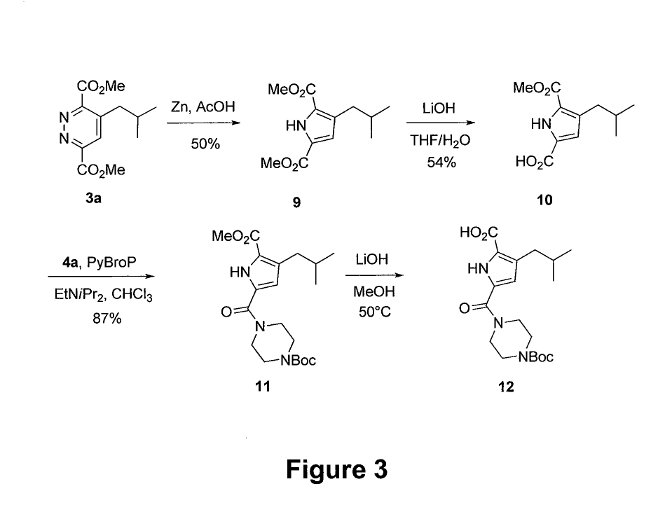

FIG. 3 is a scheme showing a strategy toward the synthesis of the OPP scaffold. The starting pyridazine 3a is reduced to the pyrrole 9 in modest yield with zinc in acetic acid and then selectively saponified to give compound 10, the carboxylic acid monoester. The coupling with the mono-protected piperazine went smoothly to give compound II in good yield. However, the saponification of the remaining methyl ester on the pyrrole ring using LiOH in MeOH was not chemoselective and other conditions of demethylation with LiI in pyridine gave low yields and failed to afford the desired carboxylic acid 12 in sufficient purity.

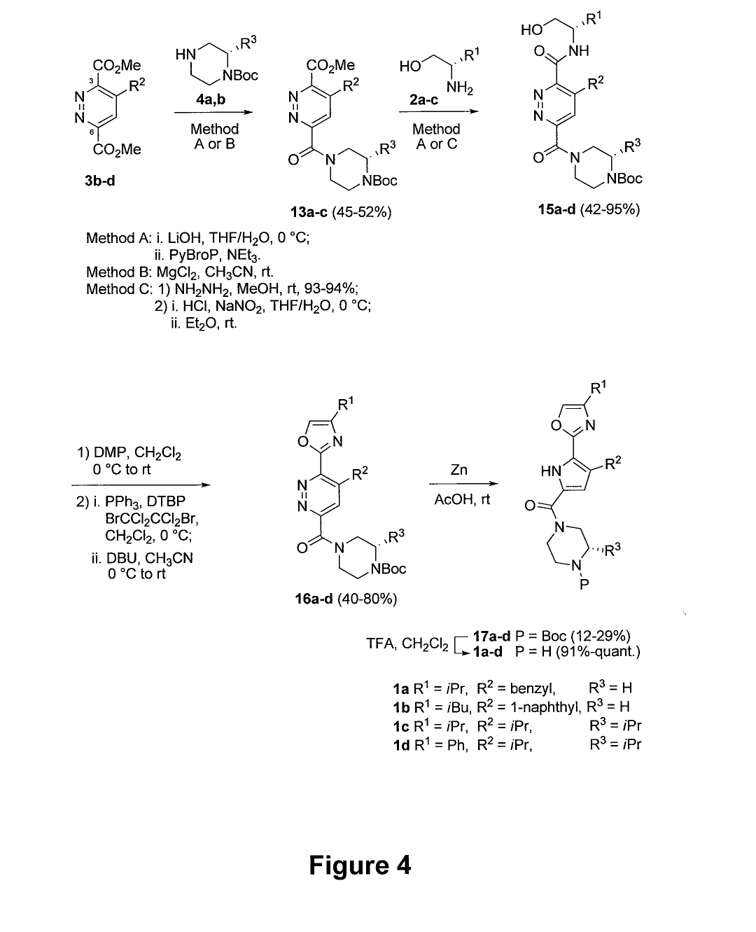

FIG. 4 is a scheme showing one synthetic route for the synthesis of the OPP scaffold. The starting material is the same as in FIG. 3, but the pyrrole ring formation is put off until the very last step, except for the piperazine imino group deprotection. Alternative methods of obtaining the OPP scaffold by merely delaying the introduction of pyrrole ring formation did not give the desired scaffold. Alternative routes are not shown. Selective saponification of starting material 3b-d is possible by one of two different methods and coupling with the piperazine is done by standard conditions with PyBroP. Another method was used in the case of pyridazine 3c, where the regioselectivity of the hydrolysis was only 4/1 in favor of the desired regioisomeric carboxylic acid. Direct activation of the methyl ester using MgCl₂ in CH₃CN gave the desired adduct 13b in 45% yield as the only regioisomer. Introducing the oxazole moiety was done in steps by first forming the amide bond with an amino alcohol to give compounds 15a-d in fair to excellent yield using either Method A or Method C or the Curtius method. Oxidation was done without complication using Dess-Martin periodinane to give aldehydes that were then exposed to oxazole formation conditions to give the desired oxazoles 16a-d. The reduction of the pyridazine with zinc in acetic acid gave the pyrrole-containing products in only 12-29% yields. Final removal of the Boc group in CH₂Cl₂ with TFA is straightforward giving the alpha-helix mimetics 1a-d in 91% to quantitative yields.

All methods comprise administering to the protein, cell, or patient in need of such treatment an effective amount of one or more compounds of the invention. Further, the compounds of the present invention may be combined with other agents to treat diseases or conditions which are modulated through disruption of the alpha helix.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

In addition to the syntheses as discussed herein, the compounds of the present invention may be prepared using conventional synthetic methods analogous to those established in the art, and, if required, standard separation or isolation techniques. All starting materials are either commercially available, or can be conventionally prepared from known starting materials without undue experimentation.

One of ordinary skill in the art will recognize that some of the compounds of Formula I can exist in different geometrical isomeric forms. While one embodiment of the present invention is limited to the described stereochemistry for the R³ moiety attached to the piperazine ring as shown in Formula II, and is preferred for interactions with the α helix, racemic and other isomeric forms of Formula II are also contemplated in the present invention. Additionally, other asymmetric atoms may be present and these compounds are thus capable of existing in the form of optical isomers, as well as in the form of racemic or nonracemic mixtures thereof, and in the form of diastereomers and diastereomeric mixtures inter alia. All of these compounds, including cis isomers, trans isomers, diastereomeric mixtures, racemates, nonracemic mixtures of enantiomers, substantially pure, and pure enantiomers, are within the scope of the present invention.

In one embodiment, the compounds comprise substantially pure enantiomers of the compound of Formula II contain no more than 5% w/w of the corresponding opposite enantiomer at R³, preferably no more than 2%, most preferably no more than 1%.

In another embodiment, additional stereocenters are present and a substantially pure enantiomers containing no more than 5% w/w of the corresponding opposite enantiomer, preferably no more than 2%, most preferably no more than 1% is within the scope of the present invention.

The optical isomers can be obtained by resolution of the racemic mixtures according to conventional processes, for example, by the formation of diastereomeric salts using an optically active acid or base or formation of covalent diastereomers. Examples of appropriate acids include, but are not limited to, tartaric, diacetyltartaric, dibenzoyltartaric, ditoluoyltartaric and camphorsulfonic acid. Mixtures of diastereomers can be separated into their individual diastereomers on the basis of their physical and/or chemical differences by methods known to those skilled in the art, for example, by chromatography or fractional crystallization. The optically active bases or acids are then liberated from the separated diastereomeric salts.

A different process for separation of optical isomers involves the use of chiral chromatography (e.g., chiral HPLC or SFC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivatization, are also useful. The optically active compounds of Formulas I-II can likewise be obtained by utilizing optically active starting materials in chiral syntheses processes under reaction conditions which do not cause racemization.

In addition, one of ordinary skill in the art will recognize that the compounds can be used in different enriched isotopic forms, e.g., enriched in the content of ²H, ³H, ¹¹C, ¹³C and/or ¹⁴C. In one particular embodiment, the compounds are deuterated. Such deuterated forms can be made by the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] (2000), 110 pp. CAN 133:68895 AN 2000:473538 CAPLUS; Kabalka, George W.; Varma, Rajender S. The Synthesis of Radiolabeled Compounds via Organometallic Intermediates. Tetrahedron (1989), 45(21), 6601-21, CODEN: TETRAB ISSN:0040-4020. CAN 112:20527 AN 1990:20527 CAPLUS; and Evans, E. Anthony. Synthesis of radiolabeled compounds, J. Radioanal. Chem. (1981), 64(1-2), 9-32. CODEN: JRACBN ISSN:0022-4081, CAN 95:76229 AN 1981:476229 CAPLUS.

The present invention also relates to useful forms of the compounds as disclosed herein, including free base forms, as well as pharmaceutically acceptable salts. Pharmaceutically acceptable salts include those obtained by reacting the main compound, functioning as a base, with an inorganic or organic acid to form a salt, for example, but not limited to, salts of hydrochloric acid, sulfuric acid, phosphoric acid, methanesulfonic acid, camphorsulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by reacting the compounds of the invention with the appropriate base via a variety of known methods.

The following are further non-limiting examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

In one embodiment, the pharmaceutically acceptable salt is an ammonium salt. Ammonium salts include alkylammonium salts such as monoalkylammonium salts, dialkylammonium salts, trialkylammonium salts, and tetraalkylammonium salts, where the salts contain 1-12, or more particularly 1-6 carbon atoms in each alkyl group. The alkyl groups may be cycloalkyl or hydroxyalkyl groups. Ammonium salts include, for example, C₁₋₆ enediamines, C₁₋₆ hydroxyalkyl or aralkyl)alkylammonium bases, such as methylamine, diethylamine, triethylamine, dicyclohexylamine, triethanolamine, ethylenediamine, tris-(hydroxymethyl)-aminomethane or benzyltrimethylammonium hydroxide.

Preferably, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

One of ordinary skill in the art will also recognize that some of the compounds of Formula I can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

One of ordinary skill in the art will further recognize that compounds of Formula I can exist in different solvate forms. Solvates of the compounds of the invention may also form when solvent molecules are incorporated into the crystalline lattice structure of the compound molecule during the crystallization process. For example, suitable solvates include hydrates, e.g., monohydrates, dihydrates, sesquihydrates, and hemihydrates.

The compounds of the invention can be administered alone or as an active ingredient of a formulation. Thus, the present invention also includes pharmaceutical compositions of one or more compounds of Formula I containing, for example, one or more pharmaceutically acceptable excipients.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

Administration may be accomplished according to patient needs, for example, orally, nasally, parenterally (subcutaneously, intravenously, intramuscularly, intrasternally and by infusion) by inhalation, rectally, vaginally, topically and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable excipients known in the art, including but not limited to suspending agents, solubilizers, buffering agents, binders, disintegrants, preservatives, colorants, flavorants, lubricants and the like. The excipients as used herein may also include carriers and/or diluents (such as sucrose, mannitol, lactose, starches). Time release capsules, tablets and gels are also advantageous in administering the compounds of the present invention.

Various liquid oral dosage forms can also be used for administering compounds of the inventions, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, paste, foam, or spray formula containing, in addition to the active ingredient, such suitable excipients as are known in the art.

For topical administration, the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the compounds according to the invention can be administered by inhalation in the form of a powder (e.g., micronized) or in the form of atomized solutions or suspensions. The aerosol formulation can be placed into a pressurized acceptable propellant.

The dosages of the compounds of the present invention depend upon a variety of factors including the particular syndrome to be treated, the severity of the symptoms, the route of administration, the frequency of the dosage interval, the particular compound utilized, the efficacy, toxicology profile, pharmacokinetic profile of the compound, and the presence of any deleterious side-effects, among other considerations. One of ordinary skill in the art of treating such diseases will be able, without undue experimentation and in reliance upon personal knowledge and the disclosure of this Application, to ascertain a therapeutically effective amount of the compounds of the present invention for a given disease.

In carrying out the procedures of the present invention, it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

Libraries of the non-peptidic alpha-helix mimetics of the present invention may be made as described herein. The libraries may be made by parallel, or combinatorial synthesis. Parallel, or combinatorial, synthesis has as its primary objective the generation of a library of diverse molecules which all share a common feature, referred to throughout this description as a scaffold. By substituting different moieties at each of the variable parts of the scaffold molecule, the amount of space explorable in a library grows. Theories and modern medicinal chemistry advocate the concept of occupied space as a key factor in determining the efficacy of a given compound against a given biological target. By creating a diverse library of molecules, which explores a large percentage of the targeted space, the odds of developing a highly efficacious lead compound increase dramatically.

Parallel synthesis is generally conducted on a solid phase support, such as a polymeric resin. The scaffold, or other suitable intermediate is cleavably tethered to the resin by a chemical linker. Reactions are carried out to modify the scaffold while tethered to the particle. Variations in reagents and/or reaction conditions produce the structural diversity, which is the hallmark of each library.

Parallel synthesis of “small” molecules (non-oligomers with a molecular weight of 200-1000) was rarely attempted prior to 1990. See, for example, Camps. et al., Annaks de Quimica, 70: 848 (1990). Recently, Ellmann disclosed the solid phase-supported parallel (also referred to as “combinatorial”) synthesis of eleven benzodiazepine analogs along with some prostaglandins and beta-turn mimetics. These disclosures are exemplified in U.S. Pat. No. 5,288,514. Another relevant disclosure of parallel synthesis of small molecules may be found in U.S. Pat. No. 5,324,483. This patent discloses the parallel synthesis of between 4 and 40 compounds in each of sixteen different scaffolds. Chen et al. have also applied organic synthetic strategies to develop non-peptide libraries synthesized using multi-step processes on a polymer support. (Chen et al., J. Am. Chem. Soc., 116: 2661-2662 (1994)).

Once a library of unique compounds is prepared, the preparation of a library of non-peptidic alpha-helix mimetics can be prepared using the library of, for example, piperazine compounds as a starting point and using the methods described herein.

In this invention, libraries containing the non-peptidic alpha-helix mimetics of the present invention are disclosed. Once assembled, the libraries of the present invention may be screened to identify individual members having bioactivity. Such screening of the libraries for bioactive members may involve, for example, evaluating the binding activity of the members of the library or evaluating the effect the library members have on a functional assay. Screening is normally accomplished by contacting the library members (or a subset of library members) with a target of interest, such as, for example, an antibody, enzyme, receptor or cell line. Library members which are capable of interacting with the target of interest are referred to herein as “bioactive library members” or “bioactive mimetics.” For example, a bioactive mimetic may be a library member which is capable of binding to an antibody or receptor, which is capable of inhibiting an enzyme, or which is capable of eliciting or antagonizing a functional response associated, for example, with a cell line. In other words, the screening of the libraries of the present invention determines which library members are capable of interacting with one or more biological targets of interest. Furthermore, when interaction does occur, the bioactive mimetic (or mimetics) may then be identified from the library members. The identification of a single (or limited number) of bioactive mimetic(s) from the library yields non-peptidic alpha-helix mimetics which are themselves biologically active, and thus useful as diagnostic, prophylactic or therapeutic agents, and may further be used to significantly advance identification of lead compounds in these fields.

Screening for the binding activity of the compound may be accomplished by an appropriate assay, such as by the calorimetric assay disclosed by Lam et al. (Nature 354:82-84, 1991) or Griminski et al. (Biotechnology 12:1008-1011, 1994). In a preferred embodiment, the library members are in solution and the target is immobilized on a solid phase. Alternatively, the library may be immobilized on a solid phase and may be probed by contacting it with the target in solution.

When the following abbreviations are used throughout this disclosure, they have the following meaning:

Ac acetyl

aq aqueous

Boc tert-butyloxycarbonyl

(Boc)₂O di-tert-butyldicarbonate

ESI electrospray (mass spectrometry)

EtOAc ethyl acetate

HPLC high-performance liquid chromatography

LC-MS liquid chromatography/mass spectroscopy

mg milligram(s)

mL milliliter

min minute(s)

mmol millimole(s)

mol mole

MS mass spectrometry

Ph phenyl

ppm parts per million

PyBrooP bromo-tris-pyrrolidone-phosphonium hexafluorophosphate

rt room temperature

SFC supercritical fluid chromatography

THF tetrahydrofuran

TLC thin layer chromatography

TOF time of flight (mass spectrometry)

w/w weight per unit weight

The present invention will now be further described by way of the following non-limiting examples. In applying the disclosure of these examples, it should be kept clearly in mind that other and different embodiments of the methods disclosed according to the present invention will no doubt suggest themselves to those of skill in the relevant art.

In the following examples, certain steps are described having low yields, not having the desired chemoselectivity, failing to afford a clean product, etc. While such information is provided to demonstrate some preferred methods for synthesizing the exemplified compounds, it is understood that theses steps may still be used. Alternatively, similar steps for producing additional compounds of the present invention according to these teachings may provide sufficient yield, selectivity, etc. for other starting materials or modified conditions.

The entire disclosures of all applications, patents and publications, cited above and below, are hereby incorporated by reference in their entirety.

### EXAMPLES

### Example 1

### Intermediate and Product Preparation

Tetrazine 5 was prepared according to the procedure described by Boger et al (Boger, D. L.; et al. Org. Synth. 1992, 70, 79). Alkynes, enamines and enol ethers are well known dienophiles for the reaction with tetrazine derivatives (Gnichtel, H.; Gumprecht, C. Liebigs Ann. Chem. 1985, 3, 628; Boger, D. L.; Sakya, S. M. J. Org. Chem. 1988, 53, 1415; Soenen, D. R.; et al. J. Org. Chem. 2003, 68, 3593). Benzyl or isobutyl groups were introduced using the corresponding commercially available alkynes 6a and 6b which gave the pyridazines 3a (Moisan, L.; et al. Heterocycles 2007, 73, 661) and 3b in good yields (Table 1). A large aromatic side chain such as naphthalene, often used to mimic the indole containing side chain of a tryptophane residue (compound 3c (Mann, E.; et al. Tetrahedron Lett. 2008, 49, 903-905)) was also prepared. In this case we used enol ether 7, obtained by methylenation with the Tebbe reagent (Tebbe reagent: μ-Chlorobis(cyclopentadienyl)-(dimethylaluminum)-μ-methylenetitanium; Tebbe, F. N.; et al. J. Am. Chem. Soc. 1978, 100, 3611; Pine, S. H.; et al. J. Am. Chem. Soc. 1980, 102, 3270) of the methyl 1-naphthaleneacetate. Short side chains were also desirable but most of the corresponding alkynes are gases and are not conveniently handled. For these, we introduced the isopropyl group to mimic a valine residue (compound 3d (Moisan, L.; et al. Heterocycles 2007, 73, 661) by using the addition of enamine 8 obtained by the condensation of isovaleraldehyde and pyrrolidine (Stork, G.; et al. J. Am. Chem. Soc. 1963, 85, 207).

With this series of substituted pyridazines in hand, we next examined their reduction to the pyrrole. This synthetic transformation usually gives the expected pyrrole in yields ranging from 27% to 70% (Hamasaki, A.; et al. J. Am. Chem. Soc. 2005, 127, 10767; Boger, D. L.; Hong, J. J. Am. Chem. Soc. 2001, 123, 8515; Boger, D. L.; Patel, M. J. Org. Chem. 1988, 53, 1405; Boger, D. L.; et al. J. Org. Chem. 1984, 49, 4405; Naud, S.; et al. Eur. J. Org. Chem. 2007, 3296). Starting from pyridazine 3a, pyrrole 9 could be obtained in 50% yield using an excess of zinc in acetic acid. We intended to introduce the piperazine moiety by a standard peptide coupling from the acid. The hydrolysis of the methyl esters in 9 with LiOH (Scheme 1) at rt was quite slow but after several days, pyrrole 10 was isolated in 54% yield. This coupled with piperazine 4a (R³═H) using PyBroP in good yield to give compound 11.

Subsequent modifications on the remaining methyl ester to introduce the oxazole moiety were problematic: saponification with LiOH in MeOH was not chemoselective, and other conditions such as demethylation with LiI in hot pyridine gave low yields and failed to afford compound 12 cleanly.

A strategy that postponed the reduction step to the pyrrole to a more advanced stage fared little better. The selective saponification/peptide coupling sequence was used on pyridazine 3 as described earlier (Moisan, L.; et al. Heterocycles 2007, 73, 661). The methyl ester in the 3-position of the pyridazine 3b is more sterically hindered and selective saponification of the methyl ester in the 6-position was possible, with one equivalent of LiOH in a THF/water mixture at 0° C. This gave clean hydrolysis of the ester to the corresponding carboxylic acid. The piperazine was then coupled under standard peptide coupling conditions with PyBroP in 52% yield over the two steps to give pyridazine-piperazine 13a (Method A, Scheme 2). In the case of pyridazine 3c, the regioselectivity of the hydrolysis was only 4/1 in favor of the desired carboxylic acid, therefore the coupling was performed by direct activation of the methyl ester using MgCl₂ in CH₃CN (González-Gómez, J. C.; et al. Tetrahedron 2003, 59, 8171) (Method B) to afford adduct 13b in 45% yield as the only regioisomer. The scope of the reaction was then extended to substituted piperazines as pyridazine 3d was coupled with 4b (R³=iPr) following Method A to give the pyridazine-piperazine adduct 13c in 52% yield over the two steps.

Introduction of the oxazole moiety was then performed by coupling an amino alcohol on the remaining ester. Hydrolysis of ester 13a in the presence of LiOH followed by a peptide coupling with L-valinol mediated by PyBroP gave amido alcohol 15a in 42% yield. Improved yields for this coupling step were obtained by using the Curtius method (Method C) (J. Meienhofer, in The peptides, Analysis, Synthesis, Biology, (Eds: E. Gross, J. Meienhofer), Academic Press, Inc., 1979; vol. 1, pp. 197-228.). Methyl esters 13b,c were first converted to the hydrazides 14b,c with hydrazine, diazotized to give the corresponding acyl azides, then reacted with amino alcohols 2a-c (R¹=iPr, R¹=iBu or R¹=Ph) to give the desired amidoalcohols 15b-d in good yields.

Oxidation of the alcohols 15a-d with Dess-Martin periodinane proceeded smoothly to give the corresponding aldehydes that were directly exposed to the oxazole formation conditions (PPh₃, 2,6-di-tert-butyl pyridine (DTBP), dibromo-tetrachloroethane and then DBU) (Wipf, P.; Lim, S. J. Am. Chem. Soc. 1995, 117, 558) to give the desired oxazoles 16a-d in good yields over the two steps. The stage was thus set to reduce the pyridazine ring into the corresponding pyrrole. Using the usual conditions with an excess of zinc in acetic acid, we obtained the desired compounds 17a-d in only 12-29% yields; these are lower than what has been observed previously (Hamasaki, A.; et al. J. Am. Chem. Soc. 2005, 127, 10767; Boger, D. L.; Hong, J. J. Am. Chem. Soc. 2001, 123, 8515; Boger, D. L.; Patel, M. J. Org. Chem. 1988, 53, 1405; Boger, D. L.; et al. J. Org. Chem. 1984, 49, 4405; Naud, S.; et al. Eur. J. Org. Chem. 2007, 3296). Final removal of the Boc protecting group of compounds 17a-d was performed with TFA in CH₂Cl₂ to afford cleanly the desired α-helix mimetics 1a-d in high yields.

### Example 2

### General Methods

Solvents and reagents were of reagent-grade, purchased from commercial suppliers, and used without further purification unless otherwise stated. Substituted NBoc-protected piperazines were purchased from Anaspec. Thin-layer chromatography (TLC) was performed on Kieselgel 60 F₂₅₄ coated plates (Merck). Preparative TLC was performed on Partisil® PK6F silica gel 60 Å, coated plates 1000 μm (Whatman). ¹H and ¹³C NMR spectra were recorded on Bruker AC 250 MHz, Varian Mercury 300 MHz or Bruker DRX 600 MHz spectrometers. Chemical shifts are expressed in ppm (δ), referenced to the protio impurity of the solvent as internal standard for ¹H and ¹³C nuclei. High resolution mass spectra were recorded on an Agilent ESI-TOF mass spectrometer by Scripps Center for Mass Spectrometry.

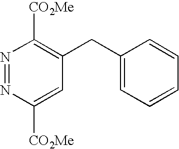

3,6-Dimethyl-4-benzylpyridazine dicarboxylate 3b; To a solution of tetrazine 5 (1.125 g, 5.7 mmol) in 1,4-dioxane (30 mL) was added 3-Phenyl-1-propyne 6b (850 μL, 6.8 mmol). The reaction vessel was sealed and heated to 90° C. for 24 h. The volatiles were evaporated under reduced pressure and the crude residue purified by silica gel chromatography (CH₂Cl₂/EtOAc 9/1) to give 3b (1.415 g, 87%) as a yellow oil. HRMS: (ESI-TOF) C₁₅H₁₄N₂O₄H⁺ expected: 287.1026, found: 287.1022.

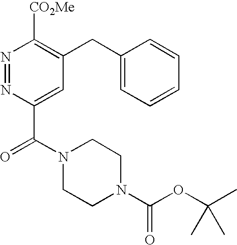

Methyl 4-benzyl-6-(4-tert-butoxycarbonyl)piperazine-1-carbonyl)pyridazine-3-carboxylate 13a; Following the Method A procedure for the piperazine coupling described below: starting from 3b (252 mg, 0.88 mmol), column AcOEt/Hexane 1/1 to 3/2, yield: 174 mg (52%). HRMS: (ESI-TOF) C₂₃H₂₈N₄O₅H⁺ expected: 441.2132. found: 441.2132.

### Method B: Typical Procedure for the Piperazine Coupling

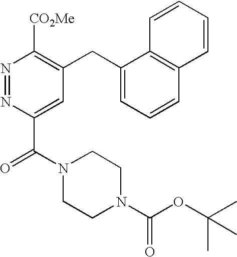

Methyl 6-(4-(tert-butoxycarbonyl)piperazine-1-carbonyl)-4-(naphthalen-1-ylmethyl)pyridazine-3-carboxylate 13b; To a stirred solution of pyridazine 3c (300 mg, 0.89 mmol) and MgCl₂ (170 mg, 1.78 mmol) in CH₃CN (5 mL) was added dropwise a solution of N-Bocpiperazine (249 mg, 1.34 mmol) in CH₃CN (3 mL) at rt. The mixture was then sonicated for 1 h and sat 24 h under nitrogen. The mixture was poured into water (10 mL) and the aqueous layer was extracted with EtOAc (3×10 mL). The organic layers were collected, dried over Na₂SO₄, filtered and concentrated in vacuo. The crude residue was purified on silica (CH₂Cl₂/AcOEt 1/0 to 8/2) to afford compound 13b (196 mg, 45%) as a yellow foam; HRMS: (ESI-TOF) C₂₇H₃₀N₄O₅H⁺ expected: 491.2289. found: 491.2280.

### Method A: Typical Procedure for the Piperazine Coupling

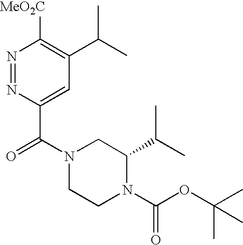

(S)-Methyl 6-(4-tert-butoxycarbonyl)-3-isopropylpiperazine-1-carbonyl)-4-isopropylpyridazine-3-carboxylate 13c; To a stirring solution of the dimethyl ester 3d (719 mg, 3.02 mmol) in THF (10 mL) at 0° C. was added dropwise a cold solution of LiOH·H₂O (139 mg, 3.3 mmol) in water (5 mL). The reaction was stirred at 0° C. until disappearance of the starting material according to TLC. The pH was then made acidic (pH 1-2) with careful addition of a 3% HCl solution and the organic phase was extracted with EtOAc (3×30 mL). The fractions were combined, dried with Na₂SO₄, filtered and the solvent was removed in vacuo. The desired monosaponified pyridazine was obtained as a pale yellow solid (510 mg, 2.27 mmol, 75%). The carboxylic acid was used directly in the next step without further purification. To a solution of the acid (340 mg, 1.5 mmol) in CH₂Cl₂ (15 mL), was added NEt₃ (0.422 mL, 3 mmol), (2S)—N-Boc-2-Isopropyl-piperazine (350 mg, 1.5 mmol) and PyBroP (707 mg, 1.5 mmol). After stirring 18 h under nitrogen, the reaction mixture was diluted with CH₂Cl₂ (50 mL) and washed with a solution of HCl (0.1 M, 10 mL) and saturated NaHCO₃ (10 mL). The organic layer was then dried with Na₂SO₄, filtered and concentrated under reduced pressure. The crude residue was purified by column chromatography (Hexane/AcOEt 1/0 to 6/4) and the desired piperazine adduct 13c was obtained as a pale yellow foam (452 mg, 1.04 mmol, 69%); HRMS: (ESI-TOF) C₂₂H₃₄N₄O₅H⁺ expected: 435.2602. found: 435.2598.

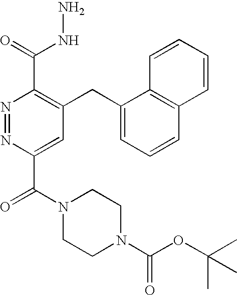

Tert-butyl 4-(6-(hydrazinecarbonyl)-5-(naphthalen-1-ylmethyl)pyridazine-3-carbonyl)piperazine-1-carboxylate 14b; Following the typical procedure for the acylhydrazide formation described below: starting from 13b (186 mg, 0.37 mmol), column CH₂Cl₂/MeOH 1/0 to 95/5, yield: 174 mg, (93%); HRMS: (ESI-TOF) C₂₆H₃₀N₆O₄H⁺ expected: 491.2401. found: 491.2388.

### Method C: Typical Procedure for the Acylhydrazide Formation

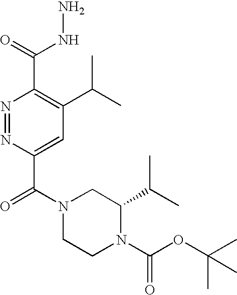

(S)-Tert-butyl 4-(6-(hydrazinecarbonyl)-5-isopropylpyridazine-3-carbonyl)-2-isopropylpiperazine-1-carboxylate 14c; To a solution of methylester 13c (224 mg, 0.51 mmol) in MeOH (10 mL), was added hydrazine hydrate (206 mg, 4.1 mmol). The reaction mixture was stirred at rt under nitrogen for 20 h, and then the volatiles removed under reduced pressure. The crude product was purified using silica gel chromatography (CH₂Cl₂/MeOH 1/0 to 95/5) to give the desired acylhydrazide 14c as a pale yellow foam (211 mg, 0.48 mmol, 94%); HRMS: (ESI-TOF) C₂₁H₃₄N₆O₄H⁺ expected: 435.2714. found: 435.2732.

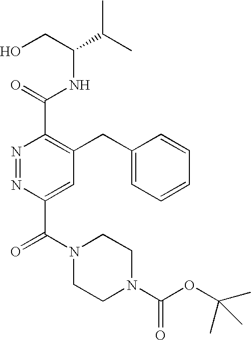

(S)-Tert-butyl 4-(5-benzyl-6-(1-hydroxy-3-methylbutane-2-ylcarbamoyl)pyridazine-3-carbonyl)piperazine-1-carboxylate 15a; To a solution of methylester 13a (275 mg, 0.624 mmol) in a THF/H₂O mixture (3/1, 4 mL) was added dropwise a solution of LiOH·H₂O (45 mg, 1.074 mmol) in H₂O (1 mL). After stirring for 1.5 h at rt the solution was poured into a solution of 0.5 M HCl and extracted four times with AcOEt. The organic phases were combined, dried over Na₂SO₄ and concentrated under reduced pressure. The residue was dissolved in CH₂Cl₂ (4 mL). L-Valinol (72 mg, 0.745 mmol, 1.2 eq.) was added followed by PyBroP (320 mg, 0.686 mmol) and iPr₂NEt (0.5 mL). The resulting mixture was stirred overnight at rt, and then a solution of 0.5 M HCl was added and the mixture was extracted several times with AcOEt. The combined organic phases were washed with H₂O, dried over MgSO₄ and concentrated under reduced pressure. The crude residue was purified by column chromatography on silica gel (AcOEt/CH₂Cl₂ 1:1) to give the diamide 15a (135 mg, 0.264 mmol, 42%) as a pale yellow oil. HRMS: (ESI-TOF) C₂₇H₃₇N₅O₅H⁺ expected 512.2867. found 512.2865.

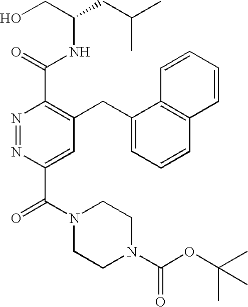

(S)-Tert-butyl 4-(6-(1-hydroxy-4-methylpentan-2-ylcarbamoyl)-5-(naphthalen-1-ylmethyl)pyridazine-3-carbonyl)piperazine-1-carboxylate 15b; Following the typical procedure for the amino alcohol coupling described below: starting from 14b (174 mg, 0.35 mmol), column CH₂Cl₂/MeOH 1/0 to 97/3, yield: 159 mg, (77%); HRMS: (ESI-TOF) C₃₂H₄₁N₅O₅H⁺ expected: 576.3180. found: 576.3169.

### Method C: Typical Procedure for the Amino Alcohol Coupling

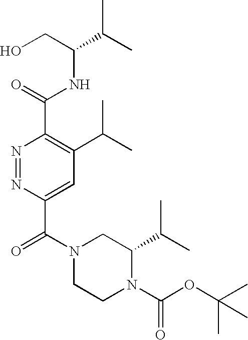

(S)-Tert-butyl 4-(6-((S)-1-hydroxy-3-methylbutane-2-ylcarbamoyl)5-isopropylpyridazine-3-carbonyl)-2-isopropylpiperazine-1-carboxylate 15c; To a solution of NaNO₂ (67 mg, 0.97 mmol), and acetic acid (87 mg, 1.45 mmol) in water (3.5 mL) at 0° C. was added dropwise a HCl solution (1 M, 1.944 mL, 1.94 mmol). After 10 min a solution of acyl hydrazide 14c (211 mg, 0.48 mmol) in THF (10 mL) was added slowly and the reaction mixture was stirred at 0° C. for 20 min. The acidic solution was made basic (pH 10) with a saturated solution of NaHCO₃ and extracted with cold Et₂O (3×15 mL). The organic fractions were collected in a second flask at 0° C., and a cold solution of L-valinol (100 mg, 0.97 mmol) in Et₂O (5 mL) was added. The reaction was allowed to warm to rt and stirred overnight. The solvent was evaporated, and the crude residue was purified by silica gel chromatography (CH₂Cl₂/MeOH 1/0 to 95/5). The desired amido-alcohol 15c was obtained as a pale yellow foam (235 mg, 0.46 mmol, 95%); (ESI-TOF) C₂₆H₄₃N₅O₅H⁺ expected: 506.3337. found: 506.3322.

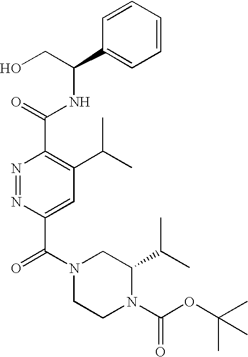

(S)-Tert-butyl 4-(6-((R)-2-hydroxy-1-phenylethylcarbamoyl)-5-isopropylpyridazine-3-carbonyl)-2-isopropylpiperazine-1-carboxylate 15d; Following Method C procedure for the amino alcohol coupling described above: starting from 14c (210 mg, 0.48 mmol), column CH₂Cl₂/MeOH 1/0 to 95/5, white foam, yield: 152 mg (58%); HRMS: (ESI-TOF) C₂₉H₄₁N₅O₅H⁺ expected: 540.3180. found: 540.3172.

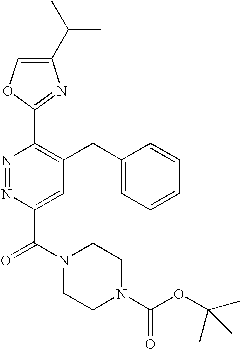

Tert-butyl 4-(5-benzyl-6-(4-isopropyloxazol-2-yl)pyridazine-3-carbonyl)piperazine-1-carboxylate 16a; Following the typical procedure for the oxazole formation described below: starting from 15a (135 mg, 0.26 mmol), column CH₂Cl₂/AcOEt 5/1 to 2/1, yield: 71 mg, (40%). HRMS: (ESI-TOF) C₂₇H₃₃N₅O₄H⁺ expected: 492.2605. found: 492.2607.

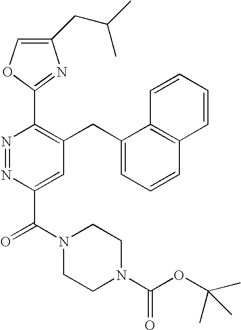

Tert-butyl 4-(6-(4-isobutyloxazol-2-yl)-5-(naphthalen-1-ylmethyl)pyridazine-3-carbonyl)piperazine-1-carboxylate 16b; Following the typical procedure for the oxazole formation described below: starting from 15b (139 mg, 0.24 mmol), column Hexane/AcOEt 1/0 to 7/3, yield: 70 mg, (52%); HRMS: (ESI-TOF) C₃₂H₃₇N₅O₄H⁺ expected: 556.2918. found: 556.2905.

### Typical Procedure for the Oxazole Formation

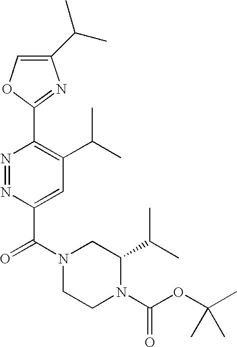

(S)-Tert-butyl 2-isopropyl-4-(5-isopropyl-6-(4-isopropyloxazol-2-yl)pyridazine-3-carbonyl)piperazine-1-carboxylate 16c; To a stirred solution of alcohol 15c (235 mg, 0.46 mmol) in CH₂Cl₂ (10 mL) at 0° C. was added Dess-Martin periodinane (296 mg, 0.69 mmol). The mixture was stirred at 0° C. for 30 min and at rt for 2 h and then washed with aqueous NaHCO₃/Na₂S₂O₃ (1:1, 10 mL), dried (Na₂SO₄), filtered and concentrated to afford the crude aldehyde. The aldehyde was then immediately dissolved in CH₂Cl₂ (6 mL) cooled to 0° C., and treated with Ph₃P (732 mg, 2.79 mmol) and 2,6-di-tert-butyl pyridine (2.055 mL, 9.3 mmol). Then BrCCl₂CCl₂Br (907 mg, 2.79 mmol) was added. After 1 h, CH₃CN (10 mL) and then DBU (1.389 mL, 9.3 mmol) were added. The mixture was then warmed to rt for 2 h and concentrated. The crude residue was purified by flash chromatography with (Hexane/AcOEt 1/0 to 7/3) to afford the desired oxazole 16c as a pale yellow solid (181 mg, 0.37 mmol, 80%); HRMS: (ESI-TOF) C₂₆H₃₉N₅O₄H⁺ expected: 486.3075. found: 486.3075.

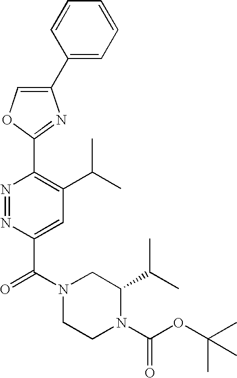

(S)-Tert-butyl 2-isopropyl-4-(5-isopropyl-6-(4-phenyloxazol-2-yl)pyridazine-3-carbonyl)piperazine-1-carboxylate 16d; Following the typical procedure for the oxazole formation described above: starting from 15d (152 mg, 0.28 mmol), column Hexane/AcOEt 1/0 to 7/3, yield: 117 mg, (80%); HRMS: (ESI-TOF) C₂₉H₃₇N₅O₄H⁺ expected: 520.2918. found: 520.2901.

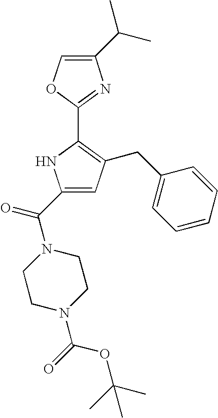

Tert-butyl 4-(4-benzyl-5-(4-isopropyloxazol-2-yl)-1H-pyrrole-2-carbonyl)piperazine-1-carboxylate 17a; Following the typical procedure for the pyridazine to pyrrole reduction described below: Starting from 16a (68 mg, 0.13 mmol), prep. TLC (CH₂Cl₂/AcOEt 3:1), yield: 8 mg, (12%); HRMS: (ESI-TOF) C₂₇H₃₄N₄O₄H⁺ expected: 478.2653. found: 479.2666.

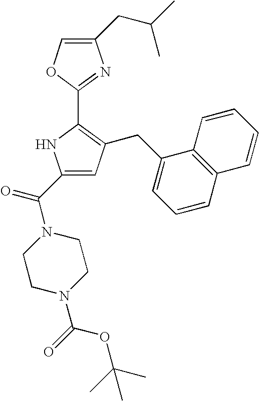

Tert-butyl 4-(5-(4-isobutyloxazol-2-yl)-4-(napthalene-1-ylmethyl)-1H-pyrrole-2-carbonyl)piperazine-1-carboxylate 17b; Following the typical procedure for the pyridazine to pyrrole reduction described below: starting from 16b (70 mg, 0.12 mmol), prep. TLC (Hexane/AcOEt 7/3), yield: 13 mg, (19%); HRMS: (ESI-TOF) C₃₂H₃₈N₄O₄H⁺ expected: 543.2966. found: 543.2976.

### Typical Procedure for the Pyridazine to Pyrrole Reduction.

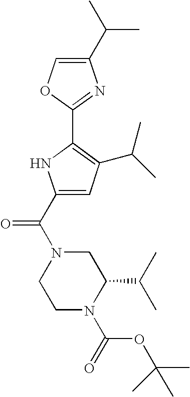

(S)-Tert-butyl-2-isopropyl-4-(4-isopropyl-5-(4-isopropyloxazol-2-yl)-1H-pyrrole-2-carbonyl)piperazine-1-carboxylate 17c; To a stirred solution of the oxazole-pyridazine-piperazine 16c (101 mg, 0.20 mmol) in CH₃CO₂H (2 mL) was added Zn dust (160 mg, 2.46 mmol). The mixture was stirred at rt for 5 h under nitrogen, and another portion of Zn dust (160 mg, 2.46 mmol) was added. After an additional 18 h under vigorous stirring, the Zn dust was removed by filtering through a plug of cotton and the residue was washed with Et₂O (2×5 mL). The filtrate and washes were combined, made basic (pH 10) with the addition of saturated sodium bicarbonate, and extracted with ether (3×10 mL). The combined ether extracts were dried over sodium sulfate and concentrated under reduced pressure. The crude residue was purified by preparative thin layer chromatography (Hexane/AcOEt 7/3) to afford the desired oxazole-pyrrole-piperazine 17c as a pale yellow solid (29 mg, 0.061 mmol, 29%); HRMS: (ESI-TOF) C₂₆H₄₀N₄O₄H⁺ expected: 473.3122. found: 473.3124.

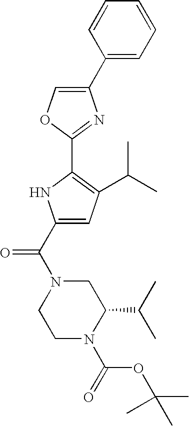

(S)-Tert-butyl 2-isopropyl-4-(4-isopropyl-5-(4-phenyloxazol-2-yl)-1H-pyrrole-2-carbonyl)piperazine-1-carboxylate 17d; Following the typical procedure for the pyridazine to pyrrole reduction described above: starting from 16d (80 mg, 0.15 mmol), Prep. TLC Hexane/AcOEt 8/2, yield: 23 mg (29%); HRMS: (ESI-TOF) C₂₉H₃₈N₄O₄H⁺ expected: 507.2966. found: 507.2954.

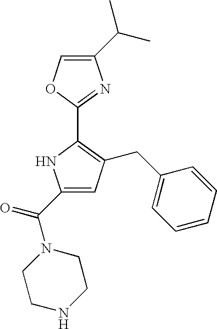

(4-Benzyl-5-(4-isopropyloxazol-2-yl-1H-pyrrol-2-yl)(piperazin-1-yl)methanone 1a; Following the typical procedure for the Boc deprotection described below: starting from 17a (7 mg, 14.6 μmol), yield: 8 mg, (quant.); HRMS: (ESI-TOF) C₂₂H₂₇N₄O₂⁺ expected: 379.2128. found: 379.2136.

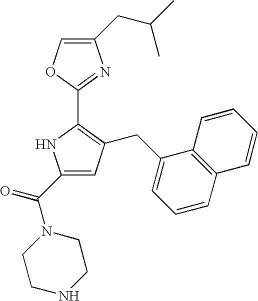

(5-(4-Isobutyloxazol-2-yl)-4-(naphthalene-1-ylmethyl)-1H-pyrrol-2-yl)(piperazin-1-yl)methanone 1b; Following the typical procedure for the Boc deprotection described below: starting from 17b (13 mg, 0.023 mmol), yield: 12 mg, (99%); HRMS: (ESI-TOF) C₂₇H₃₀N₄O₂H⁺ expected: 443.2441. found: 443.2445.

### Typical Procedure for the Boc Deprotection:

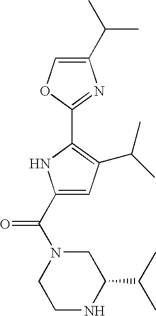

(S)-(4-Isopropyl-5-(4-isopropyloxazol-2-yl)-1H-pyrrol-2-yl)(3-isopropylpiperazin-1-yl)methanone 1c; To a stirred solution of the oxazole-pyrrole-piperazine 17c (29 mg, 0.061 mmol) in CH₂Cl₂ (4 mL) at 0° C. was added dropwise CF₃CO₂H (1 mL). After 4 h under nitrogen, the mixture was concentrated under reduced pressure. The crude residue was purified by column chromatography (CH₂Cl₂/MeOH 1/0 to 9/1) to afford the trifluoroacetic salt of the desired oxazole-pyrrole-piperazine 1c as a pale yellow solid (27 mg, 0.056 mmol, 91%); HRMS: (ESI-TOF) C₂₁H₃₂N₄O₂H⁺ expected: 373.2598. found: 373.2602.

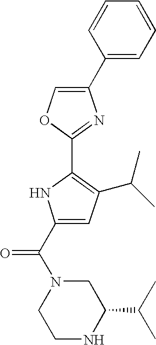

(S)-(4-Isopropyl-5-(4-phenyloxazol-2-yl)-1H-pyrrol-2-yl)(3-isopropylpiperazin-1-yl)methanone 1d; Following the typical procedure for the Boc deprotection described above: starting from 17d (23 mg, 0.48 mmol), white foam, yield: 21 mg (91%); HRMS: (ESI-TOF) C₂₄H₃₀N₄O₂H⁺ expected: 407.2441. found: 407.2426.

The preceding procedures and examples can be repeated with similar success by substituting the generically or specifically described reactants and/or operating conditions of this invention for those used in the preceding procedures and examples.

While the invention has been illustrated with respect to the production and of particular compounds, it is apparent that variations and modifications of the invention can be made without departing from the spirit or scope of the invention. Upon further study of the specification, further aspects, objects and advantages of this invention will become apparent to those skilled in the art.

What is claimed is:

1. A compound of Formula I: 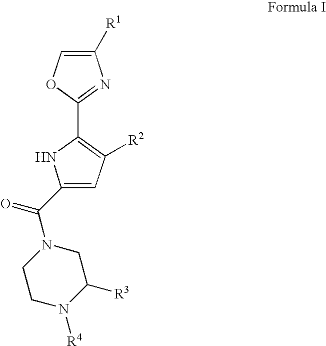
wherein R¹, R² and R³ are independently selected from the group of radicals consisting of —H, C₁-C₆ alkyl, C₆-C₁₂ aryl; C₇-C₁₈ alkylaryl, C₄-C₁₈ alkylheterocycle, C₇-C₁₈ alkylheteroaryl, wherein one —CH₂— of the alkyl may be replaced by —S—, and wherein the alkyl, aryl, or heteroaryl may be substituted with one or two moieties selected from the group consisting of C₁-C₃ alkyl, OH, SH, NH₂, and COOH;
R⁴ is selected from the group of radicals consisting of —H, —C₁-C₆ alkyl, —C(O)O(C₁-C₆ alkyl), C(O)O(C₆-C₁₂ aryl), —C(O)O(C₇-C₁₈ alkylaryl), —C(O)O(C₇-C₁₈ alkylheteroaryl), —SO₂(C₁-C₆ alkyl), —SO₂(C₆-C₁₂ aryl), —SO₂(C₇-C₁₈ alkylaryl), —SO₂(C₇-C₁₈ alkylheteroaryl), —C(O)NH(C₁-C₆ alkyl), —C(O)NH(C₆-C₁₂ aryl); —C(O)NH(C₇-C₁₈ alkylaryl), and (C₇-C₁₈ alkylheteroaryl), wherein one —CH₂— of the alkyl may be replace by —S—, and wherein the alkyl, aryl, or heteroaryl may be substituted with one or two moieties selected from the group consisting of C₁-C₃ alkyl, OH, SH, NH₂, and COOH;
or a pharmaceutically acceptable salt thereof.

2. The compound of claim 1, having the structure: 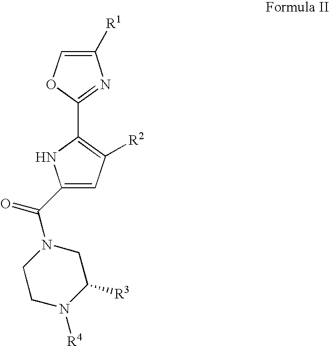
or a pharmaceutically acceptable salt thereof.

3. The compound of claim 1, wherein
R¹, R² and R³ are independently selected from the group of radicals consisting of —H, —CH₃, —CH₂CH₃, —CH(CH₃)₂, —CH₂CH₂CH₃, —CH₂CH(CH₃)₂, —CH₂CH(CH₃)CH₂CH₃, —CH(CH₃)CH₂CH₃, —CH₂CH₂CH₂CH₃, —CH₂CH₂CH(CH₃)₂, -Ph, —CH₂Ph, —CH₂CH₂Ph, —CH₂(1-naphthyl), —CH₂CH₂(1-naphthyl), —CH₂(2-naphthyl), —CH₂CH₂(2-naphthyl), —CH₂(3-indolyl), —CH₂CH₂(3-indolyl), —CH₂C₆H₄OH, —CH₂CH₂C₆H₄OH, —CH(OH)CH₃, —CH₂OH, —CH₂SH, —CH₂CH₂SH, —CH₂CH₂SCH₃, —CH₂CH₂CH₂SCH₃, —CH₂(4-imidazolyl), and —CH₂CH₂(4-imidazolyl); and
R⁴ is selected from the group of radicals consisting of —H, —(C₁-C₆ alkyl), —C(O)O(C₁-C₆ alkyl), —C(O)O(CH₂aryl), —SO₂(C₁-C₆ alkyl), —SO₂aryl, —SO₂(CH₂aryl), —C(O)NH(C₁-C₆ alkyl), —C(O)NH(CH₂aryl), and —C(O)NH(aryl).

4. The compound of claim 1, having the structure: 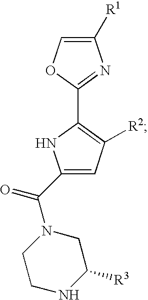
or a pharmaceutically acceptable salt thereof.

5. The pharmaceutically acceptable salt of the compound of claim 1 in the ammonium ion form.

6. The compound of claim 1, wherein R¹ is an alkyl or an aryl.

7. The compound of claim 1, wherein R² is an alkyl, aryl, or alkylaryl.

8. The compound of claim 1, wherein R³ is H or an alkyl.

9. The compound of claim 1, wherein R¹ is an alkyl or an aryl, R² is an alkyl, aryl, or alkylaryl, and R³ is H or an alkyl.

10. The compound of claim 2, having the structure: 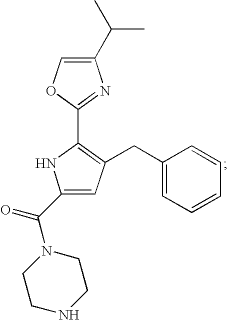
or a pharmaceutically acceptable salt thereof.

11. The compound of claim 3, having the structure: 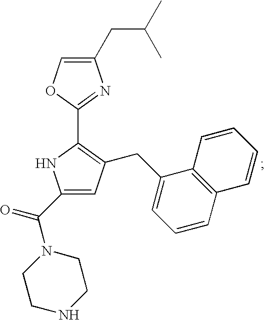
or a pharmaceutically acceptable salt thereof.

12. The compound of claim 3, having the structure: 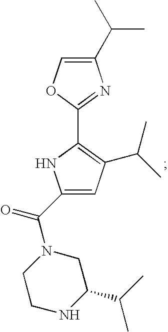
or a pharmaceutically acceptable salt thereof.

13. The compound of claim 3, having the structure: 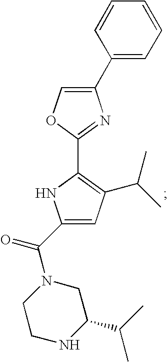
or a pharmaceutically acceptable salt thereof.

14. A library of non-peptidic alpha-helix mimetics, the library comprising a collection of compounds represented by Formula I: 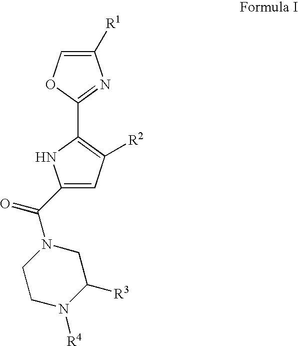
wherein R¹, R² and R³ are independently selected from the group of radicals consisting of —H, C₁-C₆ alkyl, C₆-C₁₂ aryl; C₇-C₁₈ alkylaryl, C₄-C₁₈ alkylheterocycle, C₇-C₁₈ alkylheteroaryl, wherein one —CH₂— of the alkyl may be replaced by —S—, and wherein the alkyl, aryl, or heteroaryl may be substituted with one or two moieties selected from the group consisting of C₁-C₃ alkyl, OH, SH, NH₂, and COOH;
R⁴ is selected from the group of radicals consisting of —H, —C₁-C₆ alkyl, —C(O)O(C₁-C₆ alkyl), C(O)O(C₆-C₁₂ aryl), —C(O)O(C₇-C₁₈ alkylaryl), —C(O)O(C₇-C₁₈ alkylheteroaryl), —SO₂(C₁-C₆ alkyl), —SO₂(C₆-C₁₂ aryl), —SO₂(C₇-C₁₈ alkylaryl), —SO₂(C₇-C₁₈ alkylheteroaryl), —C(O)NH(C₁-C₆ alkyl), —C(O)NH(C₆-C₁₂ aryl); —C(O)NH(C₇-C₁₈ alkylaryl), and (C₇-C₁₈ alkylheteroaryl), wherein one —CH₂— of the alkyl may be replace by —S—, and wherein the alkyl, aryl, or heteroaryl may be substituted with one or two moieties selected from the group consisting of C₁-C₃ alkyl, OH, SH, NH₂, and COOH;
or a pharmaceutically acceptable salt thereof.

15. The library of claim 14 having the structure: 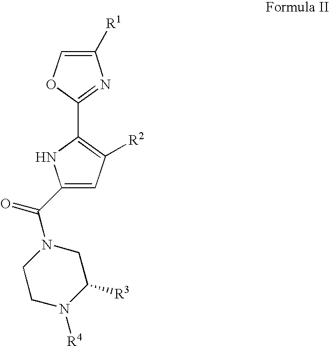
or a pharmaceutically acceptable salt thereof.

16. The library of claim 14, wherein
R¹, R² and R³ are independently selected from the group of radicals consisting of —H, —CH₃, —CH₂CH₃, —CH(CH₃)₂, —CH₂CH₂CH₃, —CH₂CH(CH₃)₂, —CH₂CH(CH₃)CH₂CH₃, —CH(CH₃)CH₂CH₃, —CH₂CH₂CH₂CH₃, —CH₂CH₂CH(CH₃)₂, -Ph, —CH₂Ph, —CH₂CH₂Ph, —CH₂(1-naphthyl), —CH₂CH₂(1-naphthyl), —CH₂(2-naphthyl), —CH₂CH₂(2-naphthyl), —CH₂(3-indolyl), —CH₂CH₂(3-indolyl), —CH₂C₆H₄OH, —CH₂CH₂C₆H₄OH, —CH(OH)CH₃, —CH₂OH, —CH₂SH, —CH₂CH₂SH, —CH₂CH₂SCH₃, —CH₂CH₂CH₂SCH₃, —CH₂(4-imidazolyl), and —CH₂CH₂(4-imidazolyl); and
R⁴ is selected from the group of radicals consisting of —H, —(C₁-C₆ alkyl), —C(O)O(C₁-C₆ alkyl), —C(O)O(CH₂aryl), —SO₂(C₁-C₆ alkyl), —SO₂aryl, —SO₂(CH₂aryl), —C(O)NH(C₁-C₆ alkyl), —C(O)NH(CH₂aryl), and —C(O)NH(aryl).

17. A pharmaceutical compound comprising the compound of claim 1 and a pharmaceutically acceptable excipient.

18. A pharmaceutical compound comprising the compound of claim 3 and a pharmaceutically acceptable excipient.

19. A method of inhibiting or disrupting the interactions between an alpha helix of a first protein and an alpha helix binding pocket of a second protein, said method comprising contacting a compound of Formula I: 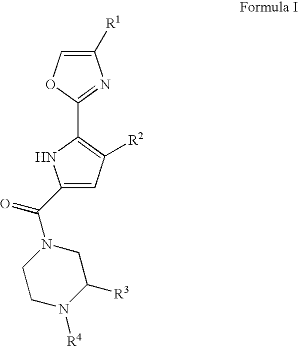
wherein R¹, R² and R³ are independently selected from the group of radicals consisting of —H, C₁-C₆ alkyl, C₆-C₁₂ aryl; C₇-C₁₈ alkylaryl, C₄-C₁₈ alkylheterocycle, C₇-C₁₈ alkylheteroaryl, wherein one —CH₂— of the alkyl may be replaced by —S—, and wherein the alkyl, aryl, or heteroaryl may be substituted with one or two moieties selected from the group consisting of C₁-C₃ alkyl, OH, SH, NH₂, and COOH;
R⁴ is selected from the group of radicals consisting of —H, —C₁-C₆ alkyl, —C(O)O(C₁-C₆ alkyl), C(O)O(C₆-C₁₂ aryl), —C(O)O(C₇-C₁₈ alkylaryl), —C(O)O(C₇-C₁₈ alkylheteroaryl), —SO₂(C₁-C₆ alkyl), —SO₂(C₆-C₁₂ aryl), —SO₂(C₇-C₁₈ alkylaryl), —SO₂(C₇-C₁₈ alkylheteroaryl), —C(O)NH(C₁-C₆ alkyl), —C(O)NH(C₆-C₁₂ aryl); —C(O)NH(C₇-C₁₈ alkylaryl), and (C₇-C₁₈ alkylheteroaryl), wherein one —CH₂— of the alkyl may be replace by —S—, and wherein the alkyl, aryl, or heteroaryl may be substituted with one or two moieties selected from the group consisting of C₁-C₃ alkyl, OH, SH, NH₂, and COOH;
or a pharmaceutically acceptable salt thereof with the first protein and the second protein under conditions wherein the interactions between the alpha helix of the first protein and the alpha helix binding pocket of the second protein are inhibited or disrupted.
